(19) United States
(12) Patent Application Publication
(10) Pub. No.: US 2025/0201340 A1
(43) Pub. Date: Jun. 19, 2025

(54) MACHINE LEARNING SYSTEM FOR PREDICTING GENE CLEAVAGE SITES BACKGROUND

(71) Applicant: The Johns Hopkins University, Baltimore, MD (US)

(72) Inventors: Alexis Battle, Baltimore, MD (US); Marios Arvanitis, Baltimore, MD (US); Joshua Popp, Baltimore, MD (US)

(21) Appl. No.: 18/866,929

(22) PCT Filed: May 18, 2023
(86) PCT No.: PCT/US2023/022770

§ 371 Date: Nov. 18, 2024

Related U.S. Application Data
(60) Provisional application No. 63/343,513, filed on May 18, 2022.

(51) Int. Cl. G16B 20/20; G06N 3/04; G16B 25/00; G16B 40/20; G16H 20/10

(52) U.S. Cl. CPC G16B 20/20; G06N 3/04; G16B 25/00; G16B 40/20; G16H 20/10

(57) ABSTRACT

Methods, systems, and computer programs for treating cancer are disclosed. In one aspect, the method includes obtaining data that represents one or more genomic variants, for each genomic variant: determining a candidate RNA sequence guide based on the genomic variant, determining feature data based on the candidate RNA sequence guide, encoding the extracted feature data into a data structure, providing the encoded data structure as an input to a machine learning model, processing the encoded data structure through each of the layers of the trained machine learning model to generate output data indicating a probability of on-target cleavage and a probability of off-target cleavage for the candidate RNA sequence guide that corresponds to the encoded data structure, obtaining output data generated by the machine learning model based on the machine learning processing the encoded data structure, and determining one or more cleavage sites based on the obtained output data.

### CROSS-REFERENCE TO RELATED APPLICATIONS

This application claims priority to U.S. Provisional Patent Application No. 63/343,513, filed on May 18, 2022, which is incorporated herein by reference in its entirety.

### BACKGROUND

Cancer is a disease that has caused human pain, suffering, and death on a large scale. Advances in computer science may be used to create novel approaches to cancer treatment.

### SUMMARY

According to one innovative aspect of the present disclosure, a cancer treatment method is disclosed. In one aspect, the method can include actions of obtaining, by one or more computers, data that represents one or more genomic variants present in genomic reads that were previously generated using a sequencing device to sequence a biological sample, for each genomic variant of the one or more genomic variants: determining, by one or more computers, a candidate RNA sequence guide based on the genomic variant, determining, by one or more computers, feature data based on the candidate RNA sequence guide, encoding, by one or more computers, the extracted feature data into a data structure, providing, by one or more computers, the encoded data structure as an input to a machine learning model that has been trained to predict a likelihood of on-target cleavage and a likelihood of off-target cleavage based on processing features extracted from a candidate RNA sequence guide, processing, by one or more computers, the encoded data structure through each of the layers of the trained machine learning model to generate output data indicating a probability of on-target cleavage and a probability of off-target cleavage for the candidate RNA sequence guide that corresponds to the encoded data structure, obtaining, by one or more computers, output data generated by the machine learning model based on the machine learning processing the encoded data structure, wherein the output data includes a probability of on-target cleavage and a probability of off-target cleavage for the candidate RNA sequence guide that corresponds to the encoded data structure, and determining, by one or more computers, one or more cleavage sites based on the obtained output data generated by the machine learning model for each of the one or more genomic variants.

Other aspects include apparatuses, systems, and computer programs for performing the actions of the aforementioned method.

The innovative method can include other optional features. For example, in some implementations, the one or more genomic variants can include one or more single nucleotide variants (SNVs) or one or more indels.

In some implementations, the plurality of genomic variants can include one or more single nucleotide variants (SNVs) and one or more indels.

In some implementations, the biological sample can include a sample of a tumor.

In some implementations, the biological sample can include a sample of a tumor and a sample of healthy tissue.

In some implementations, determining, by one or more computers, a candidate RNA sequence guide based on the genomic variant can include identifying, by one or more computers, a threshold amount of base calls that occur in the genomic read of the biological sample prior to the genomic variant.

In some implementations, the threshold amount is 20 base calls before the genomic variant.

In some implementations, determining, by one or more computers, a candidate RNA sequence guide can include determining, by one or more computers and from the set of genomic variants in a cancer sample, those variants that (i) generate a CRISPR PAM site or (ii) have more than a threshold number of base pairs difference to the non-cancer sequence.

In some implementations, determining, by one or more computers, one or more cleavage sites based on the obtained output data generated by the machine learning model for each of the one or more genomic variants can include determining, by one or more computers, one or more cleavage sites that, when cleaved, causes one or more cells of a corresponding biological sample to terminate based on the obtained output data generated by the machine learning model for each of the one or more genomic variants.

In some implementations, determining, by one or more computers, one or more cleavage sites based on the obtained output data generated by the machine learning model for each of the one or more genomic variants can include determining, by one or more computers, one or more insertion points of a suicide gene into the genomic sequence, that when expressed cause one or more cells of the biological sample to terminate, based on the obtained output data generated by the machine learning model for each of the one or more genomic variants.

These and other innovative aspects of the present disclosure are described in more detail herein in the detailed description, the accompanying drawings, and the claims.

### BRIEF DESCRIPTION OF DRAWINGS

FIG. 1 is a diagram of an example of a machine learning system for predicting DNA cleavage sites.

FIG. 2 is a flowchart of an example of a process for predicting DNA cleavage sites using a machine learning system.

FIG. 3 is a block diagram of examples of system components that can be used to implement a machine learning system for predicting DNA cleavage sites.

### DETAILED DESCRIPTION

The present disclosure is directed towards systems, methods, and computer programs for predicting a likelihood that a CRISPR (Clustered Regularly Interspaced Short Palindromic Repeats) cut at a candidate CRISPR PAM (Protospacer Adjacent Motif) site is likely to be successful. This likelihood can be expressed by referring to a likelihood of an on-target cleavage site in an entity's genome and an off-target cleavage site in an entity's genome. An on-target cleavage site is a location in the genome of an entity that is likely to result in a successful CRISPR cut at the site. In contrast, an off-target cleavage site is a location in the genome of an entity that is likely to result in an undesired CRISPR cut at the site. For purposes of this specification, a “site” is a candidate CRISPR PAM site and corresponds to a location in a genome of an entity that includes a predetermined sequence of base calls that enable a CRISPR cut to be performed.

The present disclose provides multiple technical advantages over conventional methods. First, the present disclosure provides a system and method can be used to determine which CRISPR PAM sites of multiple different CRISPR PAM sites are best suited for CRISPR cuts. In addition, the trained model of the present disclosure generates a multi-component confidence score that enables more accurate prediction of a likelihood that a CRISPR cut at a candidate CRISPR PAM site is likely be successful relative to conventional methods. These and other advantages of the present disclosure are apparent from the disclosure provided herein.

In addition, the accuracy afforded by using the present disclosure to predict successful DNA cleavage sites enables a number of genetic treatments for ailments as cancer. In some implementations, the present disclosure can be used to cut the DNA of a cell at a particular location that causes the cell containing that DNA and, e.g., a related tumor, to die. In other implementations, the present disclosure can be used to cut DNA at a particular location and then insert a suicide gene at the particular location, which causes a related tumor to die. A suicide gene can include, for example, a gene that will cause one or more cells to die through apoptosis upon its activation. In some implementations, the suicide gene may only cause death of a tumor in response to a given treatment that activates the suicide gene. In some implementations, a cellular switch that can be used to activate the suicide gene to induce apoptosis is the p53 protein. However, the present disclosure is not limited to such examples and any type of suicide gene can be used to cause the death of a tumor and suicide gene can be activated in any way to trigger apoptosis.

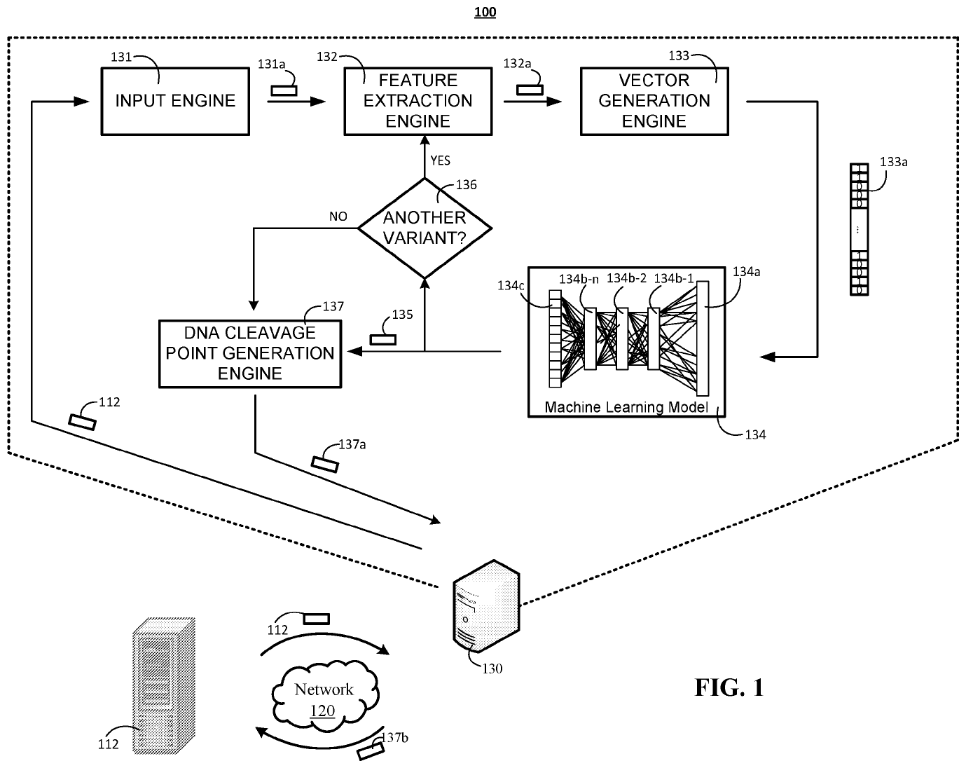

FIG. 1 is a diagram of an example of a machine learning system 100 for predicting gene cleavage sites. The system 100 can include a user device 110, a network 120, and an application server 130. The user device 110 can include, for example, any computing device that can provide input data 112 to the application server 130 via one or more networks for subsequent processing using the techniques of the present disclosure. For example, in some implementations, such a user device can receive input data 112 from a user and provide the input data 112 to the application server 130. By way of another example, in some implementations, the user device can obtain input data 112 from a database and then provide the obtained input data 112 to the application server 130. By way of yet another example, in some implementations, the user device can generate the input data 112 by, e.g., sequencing one or more biological samples and then provide the generated input data 112 to the application server 130. The one or more biological samples can include a tumor sample, a healthy tissue sample, or both.

In some implementations, the user device 110 can include a smartphone, a tablet computer, a laptop computer, a desktop computer, a nucleic acid sequencer, a server computer, or the like. In other implementations, however, the user device 110 can include a nucleic acid sequencing device. The application server 130 can include one or more computers that can receive data from the user device 110 via the network 120 and perform one or more operations on the received data. The network 120 can include one or more wired or wireless networks such as a LAN, a WAN, a wired Ethernet network, a Wi-Fi network, a cellular network, a Bluetooth network, the Internet, or any combination thereof.

The application server 130 can include an input engine 131, a feature extraction engine 132, a vector generation engine 133, a machine learning model 134, a decisioning engine 136, and a DNA cleavage point generation engine 137. In some implementations, each of the aforementioned engines can be implemented as different software and/or hardware modules on a single computer. In other implementations, the aforementioned engines may be implemented across multiple network computers. For purposes of this specification, an engine can include software instructions, hardware circuits, or a combination thereof that have been configured to realize the functionality attributed to each respective engine herein. In some implementations, such functionality can be realized by one or more hardware circuits such as one or more processors executing software instructions. In other implementations, such functionality may be realized by using program hardware logic to process data through hardware logic gates without execution of software instructions.

The input engine 131 is configured to obtain a set of input data 112 (also referred to herein as genomic data 112) from the user device via the network 120. The obtained genomic data 112 can include a set of genomic data reads, a genomic reference sequence, a set of genomic variants, or any combination thereof. Accordingly, the input engine 131 can function, at least in part, as an application programming interface (API) between the user device 110 and the application server 130. In some implementations, for example, a user device 100 such as nucleic acid sequencer can generate genomic data 112 that includes a plurality of genomic data reads by sequencing a biological sample. In some implementations, the genomic data 112 can include, for example, a FASTQ file, with the FASTQ file including data that description one or more genomic data reads, a quality score for each base call (or nucleotide) of the genomic data reads, other metadata related to the one or more data reads, or any combination or subset thereof.

Each genomic data read can correspond to a string of base calls generated by the nucleic acid sequencer based on the nucleic acid sequencer sequencing a biological sample of an entity such as a human, animal, or plant. Each base call can correspond to a nucleotide such as adenine (A), cytosine (C), thymine (T), or guanine (G) of a portion of the entity's genome. In some implementations, the genomic data 112 can also include data that identifies a reference sequence, a reference sequence, or both. The reference sequence is a sequence that corresponds to a genome of a representative entity class that is related to the entity whose sample was sequenced by the nucleic acid sequencer.

In some implementations, the input engine 131 is configured to determine a set of one or more variants based on the received genomic data 112. For example, in some implementations, the input engine 131 can be configured to perform one or more secondary analysis operations such as mapping, aligning, determination of variants, or any combination thereof, on the genomic data 112. The secondary analysis operations can be performed on the genomic data 112 in order to generate a set of one or more variants 131a. The set of one or more variants 131a can include data that identifies a set of one or positions of the entity's genome that are different from the reference sequence. The data can include the variant (e.g., the base call or nucleotide of the genomic read), the position of the variant in the entity's genome, the corresponding position of reference sequence to which the variant was aligned, data indicating a confidence score that the variant is correct, or any combination thereof. In some implementations, the genomic data 112 may not include data that identifies a reference sequence. In such implementations, the input engine 131 can determine a reference sequence for the genomic data 112. The input engine 131 can generate output data 131a that identifies each of variants identified in the genomic data 112. The output data 131a can be provide, as input, to the feature extraction engine 132.

However, the present disclosure is not limited to genomic data 112 that includes genomic data reads, a reference sequence, or a combination thereof. For example, in some implementations, the genomic data 112 can include data that identifies a set of genomic variants that were previously determined.

A genomic variant can include one or more base calls of a genomic read that are different from a corresponding location of reference sequence when the genomic read is mapped and aligned to the reference sequence. A genomic read can be Genomic variants can include single nucleotide polymorphisms (SNPs), copy number variants (CNVs), translocations, insertions, deletions, substitutions, or the like.

The output data 131a that is output by the input engine 131 and provided as an input to the feature extraction engine 132 can include data that identifies one or more variants. In some implementations, this may include genomic read data that identifies not only the variant, but also the remainder of the genomic data read itself that includes the one or more variants. The output data 131a preservers the genomic data, as a whole, that includes the one or more variants, as one or more subsequent engines will use data corresponding to one or more base calls that surround the one or more variants in later stages of the process for predicting DNA cleavage sites.

The feature extraction engine 132 can obtain the output data 131a that identifies the set of one or more variants identified in the genomic data 112 and extract a set of features related to each variant of the set. In some implementations, the feature extraction engine 132 can process the output data 131a generated by the input engine 131 to identify a candidate RNA sequence guide associated with the identified variant. Then, once the feature extraction engine 132 identifies the candidate RNA sequence guide, the feature extraction engine 132 can extract feature data 132a from the candidate RNA sequence guide. The extracted feature data 132a of the candidate RNA sequence guide for a variant provide signals to the trained machine learning model of the present disclosure that the trained machine learning model can process to make inferences regarding a likelihood of success of a candidate DNA cleavage site.

An RNA sequence guide can include a threshold amount of base calls (or nucleotides) that occur in a read immediately preceding the variant. For example, in some implementations, the RNA sequence guide can include 20 base calls (or nucleotides) of a read that occur immediately prior to a variant. The feature extraction engine 132 can identify an RNA sequence guide of a read by first identifying a location of a variant within a read, identifying a threshold number of base calls that occur immediately prior to the variant, and then transcribe the sequence of base calls (or nucleotides) of the threshold number of base calls into an RNA sequence. The RNA sequence guide of a read provides an indication of where CRISPR is to cut DNA corresponding to the read.

The feature data extracted from the RNA sequence guide for each variant of each read can include any data related to the RNA sequence guide. For example, in some implementations the extracted features of the RNA sequence guide can include data corresponding to thermodynamic features of the RNA sequence guide. Alternatively, or in addition, the extracted features of the RNA sequence guide can include, for example, data corresponding to a ratio of k-mer counts. A ratio of k-mer counts can include, for example, a percent of one or more base calls with respect to one or more other base calls in the RNA sequence guide. By way of example, a ratio of each respective base call in the RNA sequence guide or a ratio of k-mer counts. A ratio of k-mer counts in an RNA sequence guide can include, e.g., a number of occurrences of CG k-mers with respect to AT k-mers. Alternatively, or in addition, extracted features extracted of the RNA sequence guide can include data indicative of the order of nucleotides in the RNA sequence guide. Alternatively, or in addition, extracted features of the RNA sequence guide can include epigenetic data that is indicative of the location of the RNA sequence guide. Extracted features of the location of the sequence guide may include epigenetic data, conservation data, gene expression data, and any known available features of the genome at that location. The extracted feature data 132a can then be provided as an input to the vector generation engine 133.

The vector generation engine 133 can obtain the extracted feature data 132a and generate a feature vector 133a for input to the machine learning model 134. Generating the feature vector 133a can include encoding the extracted features into the feature vector 133a that numerically represents the extracted feature data 132a. The generated feature vector 133a can be defined by a feature vector vocabulary. The feature vector vocabulary can define a meaning of each field of a plurality of fields of the feature vector 133a. In some implementations, for example, feature vector vocabulary can define one or more fields of the feature vector 133a corresponding to thermodynamic features of the RNA sequence guide, one or fields corresponding of the feature vector 133a corresponding to a ratio of k-mer counts, one or more fields of the feature vector 133a corresponding to a ratio of each respective base call in the RNA sequence guide or a ratio of k-mer counts, one or more fields of the feature vector 133a corresponding to the order of nucleotides in the RNA sequence guide, one or more fields of the feature vector 133a corresponding to epigenetic data that is indicative of the location of the RNA sequence guide, or any combination thereof.

In some implementations, each feature of the feature vector 133a may have a single field that corresponds to each feature. For example, in some implementations, the feature vector 133a can have a field indicating the order of nucleotides or base calls of the RNA sequence guide. However, in other implementations, the feature vector 133a may have multiple fields for each feature or class of features. For example, for the feature of a ratio of k-mer counts, the feature vector 133a can have a field for each k-mer pair that can occur in an RNA sequence guide.

The generated feature vector 133a can then have a numerical representation of one or more of the aforementioned features associated with each field of the vector vocabulary. The generated numerical representation can function as a weight for the particular feature in the generated feature vector 133a. The generated weight can provide an indication of the presence or absence of a feature, an attribute of a feature, an extent to which the feature vector is expressed in a candidate RNA sequence guide, a frequency of occurrence of the feature, a combination thereof, or the like.

The vector generation engine 133 can provide the generated feature vector 133a as an input to the machine learning model 134.

The machine learning model 134 can be trained to process input data corresponding to features of an RNA sequence guide and generate output data 135 that indicates a likelihood of on-target cleavage and a likelihood of off-target cleavage. Once trained, the application server 130 can provide the feature vector 133a as an input to the input layer 134a of the machine learning model 134. The trained machine learning model 134 can process the feature vector 133a obtain the generated feature vector 133a and process the generated vector 133a through each hidden layer 134b-1, 134b-2, 134b-n of the trained machine learning model 134. The output of the final hidden layer 134b-n of the trained machine learning model 134 can be provided as input to the output layer 134c of the trained machine learning model 134 such as a softmax layer of the trained machine learning model 134. The output layer of the machine learning model 134 can generate output data 135 comprising a multi-component confidence score indicating a likelihood of on-target cleavage and a likelihood of off-target cleavage.

The generated output data 135 can be provided as input to the DNA cleavage point generation engine 136. The generated output data 135 can be cached in the DNA cleavage point for generation of the decisioning engine 136. The decisioning engine 136 can determine, after processing of the trained machine learning model 134, of a generated feature vector 133a, whether there is another variant for processing. If the decisioning engine 136 determines that there is another variant for processing, then the application server 130 can continue execution of system by using the feature extraction engine 132 to extract features of an RNA sequence guide associated with the variant. Alternatively, if the decisioning engine 136 determines that there is not another variant for processing, then the decisioning engine 136 can continue execution of the system by using the DNA cleavage point generation engine 136 to select one or more candidate DNA cleavage points for output.

The machine learning model 134 can be trained in a number of different ways. For example, in some implementations, the machine learning model 134 can be trained by using a database of plurality of labeled training data items. Each labeled training item of the plurality of labeled training items can include a multi-component confidence score and a label indicating whether DNA cleavage using an RNA sequence guide corresponding to the multi-component confidence score can cause cells at the DNA cleavage site to die. Cell death may be the result of DNA cleavage alone or as a result of a suicide gene inserted at the DNA cleavage site or as a result of a suicide gene inserted at the DNA cleavage site and activated by receipt of subsequent treatment by the individual. A training system can be used to process each training data item of the plurality of training data items through the machine learning model 134. After process the training data item through each layer of the machine learning model 134, the training system can compare the output data generated by the machine learning model 134 based on processing of the training data item to the label for the training data time. Then, the training system can adjust parameters of the machine learning model 134 based on the differences between the generated output of the machine learning model 134 and the label of the processed training data. This process can be iteratively performed until the differences between the output data generated by the machine learning model 134 and the label of a processed training data item satisfy a predetermine threshold. In some implementations, this can include iteratively performing the training process in order to optimize a loss function.

The DNA cleavage point generation engine (DCPGE) 136 can determine one or more cleavage sites based on the obtained output data generated by the machine learning model for each of the one or more genomic variants. This determination can include, for example, determining, by the DCPGE, one or more cleavage sites that, when cleaved, causes one or more cells of a corresponding biological sample to terminate. In some implementations, the DNA cleavage alone may be sufficient to cause the cells of the corresponding biological sample to terminate. In other implementations, however, a suicide gene may need to be inserted at the DNA cleavage site to trigger terminations of the cells of the corresponding biological sample responsive to a subsequent treatment received by an individual from which the sample was obtained.

The DCPGE 136 makes this determination based on the output data generated by the machine learning model 134 based on processing, by the trained machine learning model 134 features extracted from an RNA sequence guide for one or more variants identified by sequenced reads of a biological sample. The output data generated by the machine learning model 134 can include a multi-component confidence score. This multi-component confidence scores can include, for each set of output data generated by the trained machine learning model 134, a first score indicating a likelihood of on-target cleavage and a second score indicating a likelihood of off-target cleavage generated. The DCPGE 136 can evaluate each multi-component confidence score and then select, one or more DNA cleavage sites based on the multi-component confidences scores.

In some implementations, the DCPGE 136 can have a rules engine that applies a series one or more rules to the multi-component confidence scores. For example, the DCPGE can 136, for each multi-component confidence score of the multiple multi-component confidence score determine whether a recommendation is to be made for a DNA cleavage point at the RNA sequence guide corresponding to the multi-component confidence score by applying one or more thresholds to each score of the multi-component score associated with the RNA sequence guide. A multi-component score is associated with an RNA sequence guide if features of the RNA sequence guide were processed by the trained machine learning model 134 to generate the particular multi-component confidence score.

If, for a given RNA sequence guide, the likelihood of on-target cleavage and a likelihood of off-target cleavage each satisfy one or more predetermined thresholds, then the DCPGE 136 can output data 137a indicating that a DNA cleavage is appropriate to be made at the binding location of the RNA sequence guide associated with the multi-component confidence score. Alternatively, if, for a given RNA sequence guide, the likelihood of on-target cleavage or the likelihood of off-target cleavage do not satisfy one or more predetermined thresholds, then the DCPGE 136 can output data 137a indicating that a DNA cleavage should not be made at the binding location of the RNA sequence guide associated with the multi-component confidence score. In some implementations, the output data 137a can include data indicating whether a DNA cleavage is to occur at a single RNA sequence guide site. In other implementations, the output data 137a can include data indicating whether a DNA cleavage is to occur at each of multiple RNA sequence guide sites.

The application server 130 can provide the output data 137a to the user device 110 using the network 120. A user can read and review the output data 137a to determine one or more DNA cleavage sites.

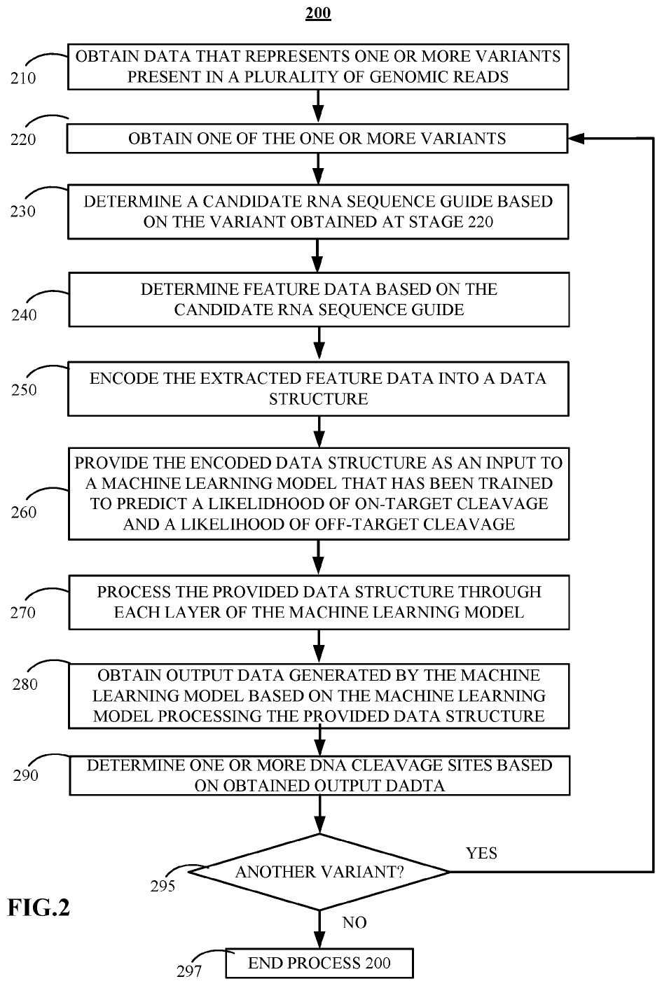

FIG. 2 is a flowchart of an example of a process 200 for predicting suicide gene insertion points using a machine learning system. For convenience, the process 200 will be described below as being performed by a system such as the system 100.

A system can begin performance of the process 200 by using one or more computers to obtain 210 data that represents one or more genomic variants present in genomic reads that were previously generated using a sequencing device to sequence a biological sample. In some implementations, for example, stage 210 can include receiving genomic variants from a secondary analysis module of a nucleic acid sequencer or secondary analysis module in communication with the nucleic acid sequencer. In other implementations, the system can access a memory device that is either local or remote that stores data representing the genomic variants. In yet other implementations, the system can obtain genomic reads generated by a nucleic acid sequencer based on the nucleic acid sequencer sequencing a biological sample and perform secondary analysis on the genomic reads to obtain the genomic variants. In some implementations, the biological sample can include a tumor sample. In other implementations, the biological sample can include a tumor sample and a healthy tissue sample.

The system can then continue execution of the process 200 by using one or more computers to obtain 220 a genomic variant received at stage 210. The system can then use one or more computers to perform stages 230-290 for each genomic variant of the received genomic variants.

The system can continue execution of the process 200 by using one or more computers to determine 230 a candidate RNA sequence guide based on the genomic variant. For example, in some implementations, the system can identify an RNA sequence guide by first identifying a location of a variant in the genome of an organism, identifying a threshold number of base calls that occur immediately prior to the variant in the genome, and then transcribe the sequence of base calls (or nucleotides) of the threshold number of base calls into an RNA sequence. The RNA sequence guide of a read provides an indication of where CRISPR is to cut DNA corresponding to the read.

The system can continue execution of the process 200 by using one or more computers to determine 240 feature data based on the candidate RNA sequence guide. For example, in some implementations, once a candidate RNA sequence guide is identified at stage 230, the system can extract feature data from the candidate RNA sequence guide. The extracted feature data of the candidate RNA sequence guide for a variant can provide signals to the trained machine learning model of the present disclosure that the trained machine learning model can process to make inferences regarding a likelihood of success of a candidate DNA cleavage site.

The feature data extracted from the RNA sequence guide for each variant of each read can include any data related to the RNA sequence guide. For example, in some implementations the extracted features of the RNA sequence guide can include data corresponding to thermodynamic features of the RNA sequence guide. Alternatively, or in addition, the extracted features of the RNA sequence guide can include, for example, data corresponding to a ratio of k-mer counts. A ratio of k-mer counts can include, for example, a percent of one or more base calls with respect to one or more other base calls in the RNA sequence guide. By way of example, a ratio of each respective base call in the RNA sequence guide or a ratio of k-mer counts. A ratio of k-mer counts in an RNA sequence guide can include, e.g., a number of occurrences of CG k-mers with respect to AT k-mers. Alternatively, or in addition, extracted features extracted of the RNA sequence guide can include data indicative of the order of nucleotides in the RNA sequence guide. Alternatively, or in addition, extracted features of the RNA sequence guide can include epigenetic data that is indicative of the location of the RNA sequence guide. The extracted feature data can be provided can then be provided as an input to the vector generation engine.

The system can continue execution of the process 200 by using one or more computers to encode 250 the extracted feature data into a data structure. In some implementations, for example, encoding the extracted feature data into a data structure can include generating a numerical representation of the feature data.

The system can continue execution of the process 200 by using one or more computers to providing 260 the encoded data structure as an input to a machine learning model that has been trained to predict a likelihood of on-target cleavage and a likelihood of off-target cleavage based on processing features extracted from a candidate RNA sequence guide. The system can continue execution of the process 200 by using one or more computers to process 270 the encoded data structure through each of the layers of the trained machine learning model to generate output data indicating a probability of on-target cleavage and a probability of off-target cleavage for the candidate RNA sequence guide that corresponds to the encoded data structure. The system can continue execution of the process 200 by using one or more computers to obtain 280 output data generated by the machine learning model based on the machine learning processing the encoded data structure. In such implementations, the output data can include a multi-component confidence score that includes a probability of on-target cleavage and a probability of off-target cleavage for the candidate RNA sequence guide that corresponds to the encoded data structure.

The system can continue execution of the process 200 by using one or more computers to determine 290 one or more DNA cleavage sites based on the obtained output data generated by the machine learning model for each of the one or more genomic variants. The output data generated by the machine learning model can include a multi-component confidence score. This multi-component confidence scores can include, for each set of output data generated by the trained machine learning model, a first score indicating a likelihood of on-target cleavage and a second score indicating a likelihood of off-target cleavage generated.

In some implementations, the system can use one or more computers to execute a rules engine that applies a series one or more rules to the multi-component confidence scores. For example, the system can, for each multi-component confidence score of the multiple multi-component confidence score, determine whether a recommendation is to be made for a DNA cleavage point at the RNA sequence guide corresponding to the multi-component confidence score by applying one or more thresholds to each score of the multi-component score associated with the RNA sequence guide.

The system can continue execution of the process 200 by using one or more computers to determine 295 whether there is another genomic variant to be evaluated using stages 230-290. Based on a determination that there are one or more additional variants to be evaluated using stages 230 to 290, the system can use one or more computers to obtain a variant at stage 220 and the continued execution of the process 200 at stage 230. Alternatively, based on a determination that there are not any additional variants to be evaluated using stages 230 to 290, the system can use one or more computers to terminate execution of the process 200 at stage 297.

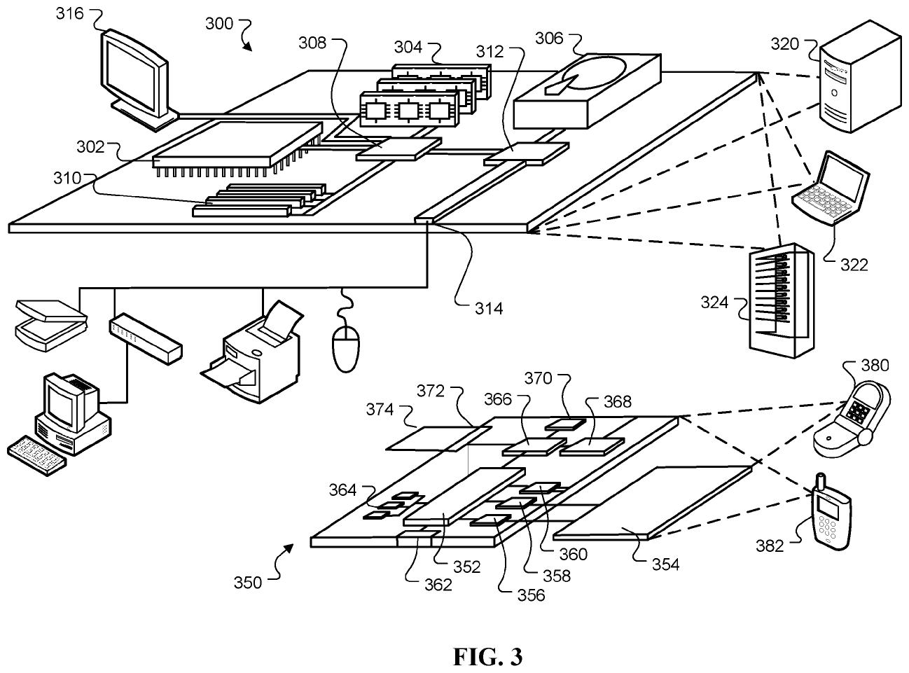

FIG. 3 is a block diagram of examples of system components that can be used to implement a machine learning system for predicting suicide gene insertion points.

Computing device 300 is intended to represent various forms of digital computers, such as laptops, desktops, workstations, personal digital assistants, servers, blade servers, mainframes, and other appropriate computers. Computing device 350 is intended to represent various forms of mobile devices, such as personal digital assistants, cellular telephones, smartphones, and other similar computing devices. Additionally, computing device 300 or 350 can include Universal Serial Bus (USB) flash drives. The USB flash drives can store operating systems and other applications. The USB flash drives can include input/output components, such as a wireless transmitter or USB connector that can be inserted into a USB port of another computing device. The components shown here, their connections and relationships, and their functions, are meant to be examples only, and are not meant to limit implementations of the inventions described and/or claimed in this document.

Computing device 300 includes a processor 302, memory 304, a storage device 306, a high-speed interface 308 connecting to memory 304 and high-speed expansion ports 310, and a low speed interface 312 connecting to low speed bus 314 and storage device 306. Each of the components 302, 304, 306, 308, 310, and 312, are interconnected using various busses, and can be mounted on a common motherboard or in other manners as appropriate. The processor 302 can process instructions for execution within the computing device 300, including instructions stored in the memory 304 or on the storage device 306 to display graphical information for a GUI on an external input/output device, such as display 316 coupled to high speed interface 308. In other implementations, multiple processors and/or multiple buses can be used, as appropriate, along with multiple memories and types of memory. Also, multiple computing devices 300 can be connected, with each device providing portions of the necessary operations, e.g., as a server bank, a group of blade servers, or a multi-processor system.

The memory 304 stores information within the computing device 300. In one implementation, the memory 304 is a volatile memory unit or units. In another implementation, the memory 304 is a non-volatile memory unit or units. The memory 304 can also be another form of computer-readable medium, such as a magnetic or optical disk.

The storage device 306 is capable of providing mass storage for the computing device 300. In one implementation, the storage device 306 can be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product can also contain instructions that, when executed, perform one or more methods, such as those described above. The information carrier is a computer- or machine-readable medium, such as the memory 304, the storage device 306, or memory on processor 302.

The high-speed controller 308 manages bandwidth-intensive operations for the computing device 300, while the low speed controller 312 manages lower bandwidth intensive operations. Such allocation of functions is only an example. In one implementation, the high-speed controller 308 is coupled to memory 304, display 316, e.g., through a graphics processor or accelerator, and to high-speed expansion ports 310, which can accept various expansion cards (not shown). In the implementation, low-speed controller 312 is coupled to storage device 306 and low-speed expansion port 314. The low-speed expansion port, which can include various communication ports, e.g., USB, Bluetooth, Ethernet, wireless Ethernet can be coupled to one or more input/output devices, such as a keyboard, a pointing device, microphone/speaker pair, a scanner, or a networking device such as a switch or router, e.g., through a network adapter. The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

The computing device 300 can be implemented in a number of different forms, as shown in the figure. For example, it can be implemented as a standard server 320, or multiple times in a group of such servers. It can also be implemented as part of a rack server system 324. In addition, it can be implemented in a personal computer such as a laptop computer 322. Alternatively, components from computing device 300 can be combined with other components in a mobile device (not shown), such as device 350. Each of such devices can contain one or more of computing device 300, 350, and an entire system can be made up of multiple computing devices 300, 350 communicating with each other.

Computing device 350 includes a processor 352, memory 364, and an input/output device such as a display 354, a communication interface 366, and a transceiver 368, among other components. The device 350 can also be provided with a storage device, such as a micro-drive or other device, to provide additional storage. Each of the components 350, 352, 364, 354, 366, and 368, are interconnected using various buses, and several of the components can be mounted on a common motherboard or in other manners as appropriate.

The processor 352 can execute instructions within the computing device 350, including instructions stored in the memory 364. The processor can be implemented as a chipset of chips that include separate and multiple analog and digital processors. Additionally, the processor can be implemented using any of a number of architectures. For example, the processor 310 can be a CISC (Complex Instruction Set Computers) processor, a RISC (Reduced Instruction Set Computer) processor, or a MISC (Minimal Instruction Set Computer) processor. The processor can provide, for example, for coordination of the other components of the device 350, such as control of user interfaces, applications run by device 350, and wireless communication by device 350.

Processor 352 can communicate with a user through control interface 358 and display interface 356 coupled to a display 354. The display 354 can be, for example, a TFT (Thin-Film-Transistor Liquid Crystal Display) display or an OLED (Organic Light Emitting Diode) display, or other appropriate display technology. The display interface 356 can comprise appropriate circuitry for driving the display 354 to present graphical and other information to a user. The control interface 358 can receive commands from a user and convert them for submission to the processor 352. In addition, an external interface 362 can be provided in communication with processor 352, so as to enable near area communication of device 350 with other devices. External interface 362 can provide, for example, for wired communication in some implementations, or for wireless communication in other implementations, and multiple interfaces can also be used.

The memory 364 stores information within the computing device 350. The memory 364 can be implemented as one or more of a computer-readable medium or media, a volatile memory unit or units, or a non-volatile memory unit or units. Expansion memory 374 can also be provided and connected to device 350 through expansion interface 372, which can include, for example, a SIMM (Single In Line Memory Module) card interface. Such expansion memory 374 can provide extra storage space for device 350, or can also store applications or other information for device 350. Specifically, expansion memory 374 can include instructions to carry out or supplement the processes described above, and can also include secure information. Thus, for example, expansion memory 374 can be provided as a security module for device 350, and can be programmed with instructions that permit secure use of device 350. In addition, secure applications can be provided via the SIMM cards, along with additional information, such as placing identifying information on the SIMM card in a non-hackable manner.

The memory can include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 364, expansion memory 374, or memory on processor 352 that can be received, for example, over transceiver 368 or external interface 362.

Device 350 can communicate wirelessly through communication interface 366, which can include digital signal processing circuitry where necessary. Communication interface 366 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 368. In addition, short-range communication can occur, such as using a Bluetooth, Wi-Fi, or other such transceiver (not shown). In addition, GPS (Global Positioning System) receiver module 370 can provide additional navigation- and location-related wireless data to device 350, which can be used as appropriate by applications running on device 350.

Device 350 can also communicate audibly using audio codec 360, which can receive spoken information from a user and convert it to usable digital information. Audio codec 360 can likewise generate audible sound for a user, such as through a speaker, e.g., in a handset of device 350. Such sound can include sound from voice telephone calls, can include recorded sound, e.g., voice messages, music files, etc. and can also include sound generated by applications operating on device 350.

The computing device 350 can be implemented in a number of different forms, as shown in the figure. For example, it can be implemented as a cellular telephone 380. It can also be implemented as part of a smartphone 382, personal digital assistant, or other similar mobile device.

Various implementations of the systems and methods described here can be realized in digital electronic circuitry, integrated circuitry, specially designed ASICs (application specific integrated circuits), computer hardware, firmware, software, and/or combinations of such implementations. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device, e.g., magnetic discs, optical disks, memory, Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device, e.g., a CRT (cathode ray tube) or LCD (liquid crystal display) monitor for displaying information to the user and a keyboard and a pointing device, e.g., a mouse or a trackball by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The systems and techniques described here can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the systems and techniques described here, or any combination of such back end, middleware, or front end components. The components of the system can be interconnected by any form or medium of digital data communication, e.g., a communication network. Examples of communication networks include a local area network (“LAN”), a wide area network (“WAN”), and the Internet.

The computing system can include clients and servers. A client and server are generally remote from each other and typically interact through a communication network. The relationship of client and server arises by virtue of computer programs running on the respective computers and having a client-server relationship to each other.

### OTHER EMBODIMENTS

A number of embodiments have been described. Nevertheless, it will be understood that various modifications can be made without departing from the spirit and scope of the invention. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

1. A cancer treatment method comprising:
obtaining, by one or more computers, data that represents one or more genomic variants present in genomic reads that were previously generated using a sequencing device to sequence a biological sample;
for each genomic variant of the one or more genomic variants:
determining, by one or more computers, a candidate RNA sequence guide based on the genomic variant;
determining, by one or more computers, feature data based on the candidate RNA sequence guide;
encoding, by one or more computers, the extracted feature data into a data structure;
providing, by one or more computers, the encoded data structure as an input to a machine learning model that has been trained to predict a likelihood of on-target cleavage and a likelihood of off-target cleavage based on processing features extracted from a candidate RNA sequence guide;
processing, by one or more computers, the encoded data structure through each of the layers of the trained machine learning model to generate output data indicating a probability of on-target cleavage and a probability of off-target cleavage for the candidate RNA sequence guide that corresponds to the encoded data structure;
obtaining, by one or more computers, output data generated by the machine learning model based on the machine learning processing the encoded data structure, wherein the output data includes a probability of on-target cleavage and a probability of off-target cleavage for the candidate RNA sequence guide that corresponds to the encoded data structure; and
determining, by one or more computers, one or more cleavage sites based on the obtained output data generated by the machine learning model for each of the one or more genomic variants.

2. The cancer treatment method of claim 1, wherein the one or more genomic variants includes one or more single nucleotide variants (SNVs) or one or more indels.

3. (canceled)

4. (canceled)

5. (canceled)

6. The method of claim 1, wherein determining, by one or more computers, a candidate RNA sequence guide based on the genomic variant comprises:
identifying, by one or more computers, a threshold amount of base calls that occur in the genomic read of the biological sample prior to the genomic variant.

7. The method of claim 3, wherein the threshold amount is 20 base calls before the genomic variant.

8. The method of claim 1, wherein determining, by one or more computers, a candidate RNA sequence guide comprises:
determining, by one or more computers and from the set of genomic variants in a cancer sample, those variants that (i) generate a CRISPR PAM site or (ii) have more than a threshold number of base pairs difference to the non-cancer sequence.

9. The method of claim 1, wherein determining, by one or more computers, one or more cleavage sites based on the obtained output data generated by the machine learning model for each of the one or more genomic variants comprises:
determining, by one or more computers, one or more cleavage sites that, when cleaved, causes one or more cells of a corresponding biological sample to terminate based on the obtained output data generated by the machine learning model for each of the one or more genomic variants.

10. The method of claim 1, wherein determining, by one or more computers, one or more cleavage sites based on the obtained output data generated by the machine learning model for each of the one or more genomic variants comprises:
determining, by one or more computers, one or more insertion points of a suicide gene into the genomic sequence, that when expressed cause one or more cells of the biological sample to terminate, based on the obtained output data generated by the machine learning model for each of the one or more genomic variants.

11. A system for treating cancer comprising:
one or more computers; and
one or more computer-readable storage devices storing instructions that, when executed by the one or more computers, cause the one or more computers to perform operations, the operations comprising:
obtaining, by the one or more computers, data that represents one or more genomic variants present in genomic reads that were previously generated using a sequencing device to sequence a biological sample;
for each genomic variant of the one or more genomic variants:
determining, by the one or more computers, a candidate RNA sequence guide based on the genomic variant;
determining, by the one or more computers, feature data based on the candidate RNA sequence guide;
encoding, by the one or more computers, the extracted feature data into a data structure;
providing, by the one or more computers, the encoded data structure as an input to a machine learning model that has been trained to predict a likelihood of on-target cleavage and a likelihood of off-target cleavage based on processing features extracted from a candidate RNA sequence guide;
processing, by the one or more computers, the encoded data structure through each of the layers of the trained machine learning model to generate output data indicating a probability of on-target cleavage and a probability of off-target cleavage for the candidate RNA sequence guide that corresponds to the encoded data structure;
obtaining, by the one or more computers, output data generated by the machine learning model based on the machine learning processing the encoded data structure, wherein the output data includes a probability of on-target cleavage and a probability of off-target cleavage for the candidate RNA sequence guide that corresponds to the encoded data structure; and
determining, by the one or more computers, one or more cleavage sites based on the obtained output data generated by the machine learning model for each of the one or more genomic variants.

12. The system of claim 11, wherein the one or more genomic variants includes one or more single nucleotide variants (SNVs) or one or more indels.

13. (canceled)

14. (canceled)

15. (canceled)

16. The system of claim 11, wherein determining, by the one or more computers, a candidate RNA sequence guide based on the genomic variant comprises:
identifying, by the one or more computers, a threshold amount of base calls that occur in the genomic read of the biological sample prior to the genomic variant.

17. The system of claim 16, wherein the threshold amount is 20 base calls before the genomic variant.

18. The system of claim 11, wherein determining, by the one or more computers, a candidate RNA sequence guide comprises:
determining, by the one or more computers and from the set of genomic variants in a cancer sample, those variants that (i) generate a CRISPR PAM site or (ii) have more than a threshold number of base pairs difference to the non-cancer sequence.

19. The system of claim 11, wherein determining, by the one or more computers, one or more cleavage sites based on the obtained output data generated by the machine learning model for each of the one or more genomic variants comprises:
determining, by the one or more computers, one or more cleavage sites that, when cleaved, causes one or more cells of a corresponding biological sample to terminate based on the obtained output data generated by the machine learning model for each of the one or more genomic variants.

20. The system of claim 11, wherein determining, by the one or more computers, one or more cleavage sites based on the obtained output data generated by the machine learning model for each of the one or more genomic variants comprises:
determining, by the one or more computers, one or more insertion points of a suicide gene into the genomic sequence, that when expressed cause one or more cells of the biological sample to terminate, based on the obtained output data generated by the machine learning model for each of the one or more genomic variants.

21. One or more computer-readable storage media storing instructions that, when executed by one or more computers, cause the one or more computers to perform operations, the operations comprising:
obtaining data that represents one or more genomic variants present in genomic reads that were previously generated using a sequencing device to sequence a biological sample;
for each genomic variant of the one or more genomic variants:
determining a candidate RNA sequence guide based on the genomic variant;
determining feature data based on the candidate RNA sequence guide;
encoding the extracted feature data into a data structure;
providing the encoded data structure as an input to a machine learning model that has been trained to predict a likelihood of on-target cleavage and a likelihood of off-target cleavage based on processing features extracted from a candidate RNA sequence guide;
processing the encoded data structure through each of the layers of the trained machine learning model to generate output data indicating a probability of on-target cleavage and a probability of off-target cleavage for the candidate RNA sequence guide that corresponds to the encoded data structure;
obtaining output data generated by the machine learning model based on the machine learning processing the encoded data structure, wherein the output data includes a probability of on-target cleavage and a probability of off-target cleavage for the candidate RNA sequence guide that corresponds to the encoded data structure; and
determining one or more cleavage sites based on the obtained output data generated by the machine learning model for each of the one or more genomic variants.

22. The computer-readable storage media of claim 21, wherein the one or more genomic variants includes one or more single nucleotide variants (SNVs) or one or more indels.

23. (canceled)

24. (canceled)

25. (canceled)

26. The computer-readable storage media of claim 21, wherein determining a candidate RNA sequence guide based on the genomic variant comprises:
identifying a threshold amount of base calls that occur in the genomic read of the biological sample prior to the genomic variant.

27. (canceled)

28. The computer-readable storage media of claim 21, wherein determining a candidate RNA sequence guide comprises:
determining, from the set of genomic variants in a cancer sample, those variants that (i) generate a CRISPR PAM site or (ii) have more than a threshold number of base pairs difference to the non-cancer sequence.

29. The computer-readable storage media of claim 21, wherein determining one or more cleavage sites based on the obtained output data generated by the machine learning model for each of the one or more genomic variants comprises:
determining one or more cleavage sites that, when cleaved, causes one or more cells of a corresponding biological sample to terminate based on the obtained output data generated by the machine learning model for each of the one or more genomic variants.

30. The computer-readable storage media of claim 21, wherein determining one or more cleavage sites based on the obtained output data generated by the machine learning model for each of the one or more genomic variants comprises:
determining one or more insertion points of a suicide gene into the genomic sequence, that when expressed cause one or more cells of the biological sample to terminate, based on the obtained output data generated by the machine learning model for each of the one or more genomic variants.
